(19) United States
(12) Patent Application Publication
(10) Pub. No.: US 2026/0293880 A1
(43) Pub. Date: Oct. 1, 2026

(54) PERFUSION SYSTEM FOR CORNEAL ENDOTHELIAL CELL GRAFT EVALUATION

(71) Applicant: REGENTS OF THE UNIVERSITY OF MINNESOTA, Minneapolis, MN (US)

(72) Inventors: Joshua Honghan Hou, Minneapolis, MN (US); Ching Yuan, St. Paul, MN (US); Sung Lee, St. Paul, MN (US); Peter Bedard, St. Paul, MN (US)

(21) Appl. No.: 19/701,379

(22) Filed: Jun. 8, 2026

Related U.S. Application Data
(62) Division of application No. 17/843,086, filed on Jun. 17, 2022.
(60) Provisional application No. 63/211,906, filed on Jun. 17, 2021.

(51) Int. Cl. A01N 1/122; A01N 1/143; C12N 5/079; G01N 33/50

(52) U.S. Cl. CPC A01N 1/122; A01N 1/143; C12N 5/0621; G01N 33/5082

(57) ABSTRACT

Compositions, devices, and systems comprising a corneal tissue carrier, wherein the corneal tissue carrier comprises a corneal tissue sample and a fluid; wherein the fluid comprises resazurin. Additionally, methods including measuring cell viability.

### CROSS-REFERENCE TO RELATED APPLICATION

This application claims the benefit of U.S. Provisional Patent Application No. 63/211,906, filed Jun. 17, 2021, which is incorporated herein by reference in its entirety.

### SUMMARY

This disclosure describes, in one aspect, a composition including a corneal tissue carrier, a corneal tissue sample, and a fluid solution including resazurin.

In one or more embodiments, the corneal tissue carrier of the composition includes a modified Jones tube.

In one or more embodiments, the corneal tissue sample includes a corneal endothelial cell graft.

In one or more embodiments the fluid solution includes a corneal preservation solution.

In one or more embodiments, the concentration of resazurin in the fluid solution is 40 μM (micromolar) to 100 μM.

In one or more embodiments, the volume of the fluid solution is 200 to 400 microliters (μL).

In another aspect, this disclosure describes methods of using a corneal tissue carrier. Generally, the methods include providing a corneal tissue carrier including a corneal tissue sample and a first fluid solution, introducing a second fluid solution including resazurin into the corneal tissue carrier, incubating the corneal tissue carrier, the corneal tissue sample, and the second fluid solution, removing the second fluid solution from the corneal tissue carrier, and collecting at least a portion of the second fluid solution.

In one or more embodiments, the corneal tissue carrier includes a modified Jones tube.

In one or more embodiments, the corneal tissue sample includes a corneal endothelial cell graft.

In one or more embodiments, the first fluid solution includes a corneal preservation solution.

In one or more embodiments, the second fluid solution includes resazurin at a concentration in a range of 40 μM to 100 μM.

In one or more embodiments, the volume of the second fluid solution introduced is a fixed volume.

In one or more embodiments, removing the second fluid solution from the corneal tissue carrier includes introducing a third fluid solution. In some of these embodiments, the third fluid solution includes a corneal preservation solution.

In one or more embodiments, the method further includes measuring the presence of resorufin in the second fluid solution. In some of these embodiments, the method includes correlating the presence of resorufin to the number of viable cells in the corneal tissue sample.

In another aspect, this disclosure describes systems including a corneal tissue carrier including an inlet, an outlet, and a body portion tapered from the inlet toward the outlet. The system includes an input valve coupled to the corneal tissue carrier inlet and configured to control input of a first fluid solution and input of a second fluid solution to the corneal tissue carrier, wherein one of the first and second fluid solutions includes resazurin. The system additionally includes an output valve coupled to the corneal tissue carrier outlet and configured to control output of fluid solution from the corneal tissue carrier, and a manipulation system coupled to the corneal tissue carrier and configured to control movement of the corneal tissue carrier.

In one or more embodiments, the corneal tissue carrier includes a modified Jones tube.

In one or more embodiments, the corneal tissue carrier stores a corneal endothelial cell graft.

In one or more embodiments, the input valve includes a first input coupled to a first fluid reservoir and a second input coupled to a second fluid reservoir and the input valve is configured to input one fluid solution to the corneal tissue carrier at a time. In some of these embodiments, the first input is coupled to a first pump, the second input is coupled to a second pump, or both.

In one or more embodiments, the system further includes a sensor system configured to monitor fluorescence of the fluid solution including resazurin in the corneal tissue carrier.

### BRIEF DESCRIPTION OF THE FIGURES

FIG. 1 illustrates the chemical reaction from resazurin to resorufin and the accompanying change from no fluorescence to fluorescence.

FIG. 2 illustrates two embodiments of the disclosed system. (A) A schematic of an illustrative embodiment of the disclosed system. (B) One embodiment of the disclosed system.

FIG. 3 shows drawings of two types of modified Jones tubes. (A) A Straiko-type modified Jones tube. (B) A LEITR-type modified Jones tube.

FIG. 4 shows a decrease in fluorescence that indicates endothelial cell loss (ECL) in two types of modified Jones tubes.

### DETAILED DESCRIPTION OF ILLUSTRATIVE EMBODIMENTS

This disclosure describes perfusion systems and methods for evaluating corneal endothelial cell grafts.

In recent years, use of pre-dissected and preloaded corneal endothelial grafts such Descemet's membrane endothelial keratoplasty (DMEK) and Descemet's stripping automated keratoplasty (DSAEK) grafts has become increasingly popular among corneal surgeons. The “injection-ready” grafts not only simplify the surgical procedure and reduce overall surgery/operating room time, but notably reduce potential tissue wastage by eliminating risk of intraoperative complications with graft loading and injection. In light of these advantages, many US eye banks now provide DMEK or DSAEK grafts prepared in many types of modified Jones tubes and injector configurations.

However, evaluation tools that can provide reliable and comprehensive graft quality assessment remain lacking. Currently, the Eye Bank Association of America's medical standards require use of at least two methods to evaluate transplant grafts prepared by eye banks. Methods for evaluating endothelial quality of processed corneal donor grafts include slit lamp examination, specular microscopy, Gabor-domain OCT and the use of vital dyes. Each of these methods include challenges and/or drawbacks. For example, slit lamp examination provides visualization of the corneal layers, but it may be difficult to examine a DMEK scroll due to optical aberration caused by the curvature of the graft. Specular microscopy provides an endothelial cell count, but it is focused on the central part of the graft (maximum 0.2 mm² area) and cannot identify cells that are dying or committed to apoptosis. Gabor-domain OCT provides a curvature-corrected image of the scrolled DMEK graft and can visualize individual cells, allowing cell counting over a larger area (maximum 1 mm²). However, this equipment does not image the entire graft and has much higher costs that may be prohibitive to eye banks. Vital dyes, such as Calcein-AM, can be used to evaluate cell viability but may only be used as an endpoint evaluation as the dyes are typically permanent and cytotoxic. In one or more embodiments, graft viability may be measured in terms of endothelial cell loss (ECL).

Resazurin is a non-toxic, cell-permeable compound that is blue in color and virtually non-fluorescent. Upon entering living cells, resazurin is reduced to resorufin, a compound that is red in color and highly fluorescent. Conversion of resazurin to resorufin requires NADH, which is readily available in viable cells but is not readily available in dead or apoptotic cells. ALAMARBLUE cell viability reagent (Trek Diagnostic Systems LLC, Cleveland, OH) is an exemplary ready-to-use resazurin-based solution that functions as a cell health indicator by using the reducing power of living cells to quantitatively measure viability.

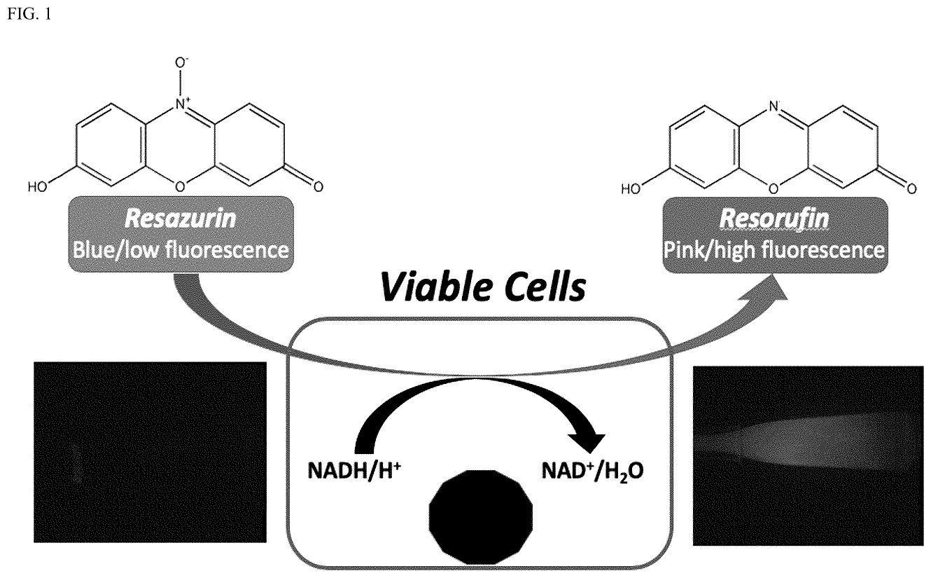

Changes in viability may be easily detected using an absorbance-based or fluorescence-based plate reader. FIG. 1 shows the chemical structures of resazurin and resorufin and the change in fluorescence observed when resazurin is converted to resorufin. Resazurin has broad applicability and can be used with various human cell types, other animal cells, bacterial cells, plant cells, and fungal cells. In the context of DMEK and/or DSAEK, resazurin can be used to determine the viability of the entirety of a corneal endothelial cell graft.

Disclosed herein is a corneal tissue carrier including a corneal tissue sample and a fluid with resazurin. Additionally disclosed herein are a perfusion system including a fluid with resazurin, and methods of determining cell viability of a corneal endothelial cell graft using resazurin.

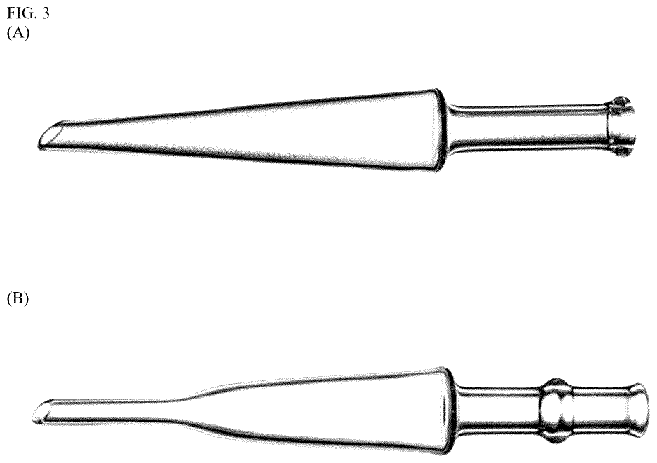

Disclosed corneal tissue carriers may include a corneal tissue sample and a fluid, wherein the fluid includes resazurin. The carrier may be a modified Jones tube as shown in FIG. 3A or FIG. 3B. The carrier may be a device similar to a modified Jones tube. The carrier may include a coating to prevent shearing of the corneal tissue sample. In one or more embodiments, the corneal tissue sample may be a corneal endothelial cell graft. The concentration of resazurin in the fluid may vary. The concentration of resazurin may be 20 μM to 200 μM, 40 μM to 100 μM, or 44 μM. The volume of fluid may be 50 μL to 600 μL, 100 μL to 500 μL, 200 μL to 400 μL, or 300 μL.

Disclosed carriers and perfusion systems may include a device that is similar to a modified Jones tube. Jones tubes are small glass tubes that are inserted in the eyelids to remove tears in patients whose tear ducts are no longer functioning. Modified Jones tubes, and other similar glass tissue carriers, are modified in shape from the original Jones tube to allow for controlled loading, storing, and ejecting of a corneal endothelial graft into the recipient anterior chamber. For example, a Descemet membrane endothelial keratoplasty (DMEK) graft may be stored and provided to doctors for use in posterior corneal graft surgery in tissue carriers such as a modified Jones tube.

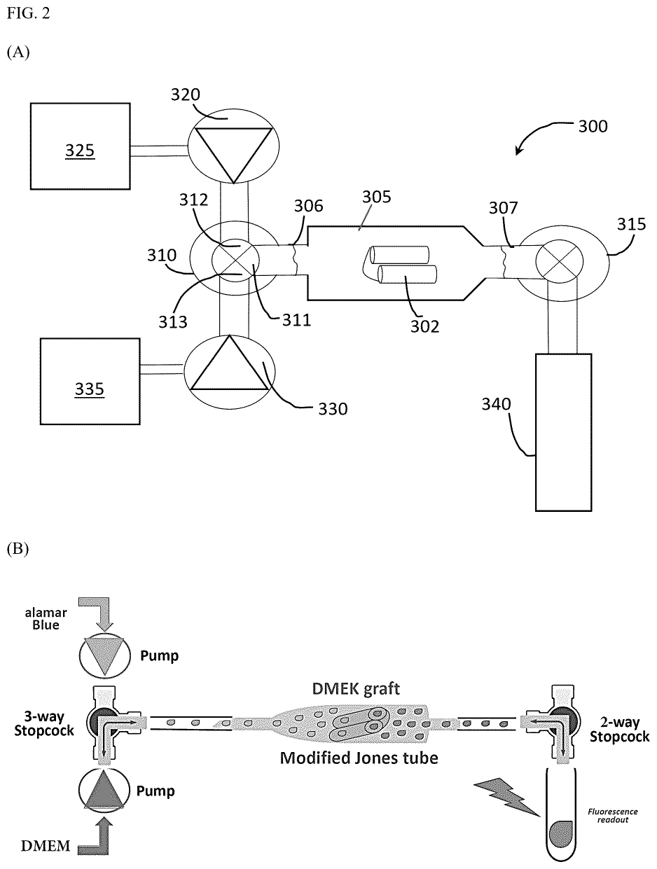

In the systems disclosed herein, a DMEK graft may be dissected and loaded into a modified Jones tube that includes corneal preservation solution. The modified Jones tube can include one or more three-way (three-way) stopcocks on both ends of the modified Jones tube. The system can further include one or more pumps (e.g., a peristaltic pump) in fluid communication with one of the three-way stopcocks. In the exemplary embodiment illustrated in FIG. 2, the system includes two three-way stopcocks (315,310), each of which is independently in fluid communication (320,330) with a peristaltic pump. When a pump is included in the system, a pump may be used to introduce a cell viability reagent (e.g., a resazurin-based reagent) into the modified Jones tube. Alternatively, or additionally, a pump may be used to introduce a culture medium (e.g., Dulbecco's Modified Eagle Medium (DMEM)) or cornea preservation medium into the modified Jones tube. In one or more embodiments, the pump may be a peristaltic pump.

In the system illustrated in FIG. 2A, a first pump 320 may be used to perfuse a cell viability reagent into the modified Jones tube as a tissue carrier 305 containing the tissue 302 (e.g., corneal endothelial cell graft) to, for example, displace corneal preservation solution from the modified Jones tube. Once the perfusion of the cell viability reagent is complete, the tissue 302 may be incubated in the cell viability reagent for a period of time. The tissue 302 may then be imaged using, for example, fluorescence microscopy. After the tissue 302 has been assessed for vitality, the cell viability reagent may be replaced with cell culture medium, such as DMEM, via the second pump 330. Furthermore, the tissue 302 may be re-perfused with storage medium via the second pump 330 if the tissue must be kept in storage for later use, as with serial testing. In one or more embodiments, the only difference between the medium fluid and the analysis fluid is that the analysis fluid contains an analysis reagent, such as a cell viability reagent, and the medium fluid does not.

The exemplary system 300 illustrated in FIG. 2A includes a tissue carrier 305 that is connected to and/or in fluid communication with a first three-way stopcock 310 and a second valve 315. The first three-way stopcock 310 has a first port 311, an analysis port 312, and a medium port 313. The first port 311 of the first three-way stopcock 310 is connected to the first end 306 of the tissue carrier 305. In one or more embodiments, the analysis port 312 of the first three-way stopcock 310 is connected to a first pump 320 that is in fluid communication with a source of analysis fluid 325; and the medium port 313 is connected to a second pump 330 that is in fluid communication with a source of medium fluid 335. In one or more embodiments, the second valve 315 may be a three-way stopcock, a two-way stopcock, or a single exit port.

In one or more embodiments, the tissue carrier 305 may be considered a modified Jones tube. Illustrative examples of tissue carriers include, for example any type of modified Jones tube, a Straiko modified Jones tube (depicted in FIG. 3A), a LEITR modified Jones tube (depicted in FIG. 3B), a DORC glass injector, a Gueder glass cannula, a modified version of any of these or other tissue carriers, or another type of modified Jones tube. In one or more embodiments, where the system 300 is to be used for storing a corneal graft, the tissue carrier 305 may have a volume of, for example, about 350 μL for example. If the system is to be used for some other procedure, the volume of the tissue carrier 305 may be different. In one or more embodiments, the tissue carrier 305 may be made of glass, plastic (e.g., acrylic, teflon, polycarbonate, or polyethylene), metal, or combinations of the aforementioned materials.

When a tissue carrier 305 is used in a corneal graft surgery, the first end 306 is generally used for ejection of the tissue (e.g., corneal endothelial cell graft) 302 when it is being ejected for placement in a patient. In one or more embodiments, the tissue carrier is a modified Jones tube. In some of these embodiments during perfusion, the analysis fluid or medium fluid may flow counter to the direction that the tissue is typically ejected from the tissue carrier 305 during placement of the tissue 302 in a patient. For example, the analysis fluid may flow from the first end 306 to the second end 307 during testing, but the tissue 302 may be ejected for placement in a patent through the first end 306.

In one or more embodiments, the tissue carrier 305 is a DORC or Guerder glass injector. In some of these embodiments, the perfusion of analysis fluid or medium fluid may flow in the same direction that the tissue 302 is typically ejected from the tissue carrier 305 during placement of the tissue 302 in a patient. For example, the analysis fluid may flow from the second end 307 to the first end 306, and the tissue 302 may be ejected for placement in a patient through the second end 307. Fluid direction should be designed according to specifications of the tissue carrier used

The tissue carrier 305 may be connected to the first three-way stopcock 310 and the second valve 315 using any type of connective tubing for example. The particular choice of size, material, etc. may depend at least in part on the particular tissue that will be used in the system and the particular procedure that the system is being used for, as well as other possible considerations. For example, when the system 300 is to be used for graft viability assessment, connective tubing connects the tissue carrier 305 to the first three-way stopcock 310 and the second valve 315; the first three-way stopcock 310 to the first pump 320 and the second pump 330; the first pump 320 and the second pump 330 to the sources of fluid; and the tissue carrier 305 to the second valve 315; and the second valve 315 to the analysis container 340. Connective tubing may include, for example, silicone tubing, polyethylene (PE) tubing, rubber tubing, etc. In one or more embodiments, the connective tubing may have outer dimensions from 0.5 to 3.5 mm, or from 0.8 to 3.2 mm. In an embodiment where the system 300 is to be used for a graft viability assessment, the total volume of the system between the first three-way stopcock 310 and the second valve 315 may be about 500 μL for example.

It should also be noted that the first pump and the second pump may in fact be one single pump that is only connected to the relevant connective tubing when the first three-way stopcock is manipulated in such a way to allow access to the particular port on the first three-way stopcock.

In one or more embodiments, an analysis container 340 may also be provided. The analysis container 340 may be configured to be immediately placed into an analytical instrument for measuring fluorescence of a sample, absorbance of a sample, or another analytical measurement. Alternatively, the analysis container 340 may be configured to merely collect fluid that may then be introduced into a user specific analysis container that is configured to go with a user-specific analytical instrument of the user's choice. The type of analysis container may generally be chosen based on a number of different factors, including for example, the type of tissue 302 being housed in the tissue carrier 305, the relevant volume of the tissue carrier 305 and the overall system, different types of analytical instrumentation used by potential users, relevant volume necessary for the particular analysis being undertaken on the tissue 302, other factors not presented herein, or some combination thereof.

An example of an embodiment of the disclosed system is shown in FIG. 2B. In this system, the medium fluid is DMEM, and the analysis fluid contains the cell viability reagent (e.g., ALAMARBLUE, Trek Diagnostic Systems LLC, Cleveland, OH). The three-way stopcock 310 is connected to source of medium fluid 335 via a peristaltic pump and the source of analysis fluid 325 via a second peristaltic pump. The tissue carrier 305 is a modified Jones tube, and the tissue 302 is a DMEK graft. The modified Jones tube is connected by the second end 307 to a two-way stopcock, which is the second valve 315. An analysis container 340 is provided in the form of a tube.

In one or more embodiments, disclosed systems may be provided to users as a kit of parts. For example, a user may be provided with a tissue carrier, either with or without an already introduced tissue and a first fluid; a first three-way stopcock, and a second valve. As another example, a user may be provided with a tissue carrier 305, either with or without an already introduced tissue 302 and a first fluid; a first three-way stopcock; a second valve; and tubing for connecting the tissue carrier 305 to the first three-way stopcock and the second valve. As another example, a user may be provided with a tissue carrier 305, either with or without an already introduced tissue 302 and a first fluid; a first three-way stopcock; a second valve; tubing for connecting the tissue carrier 305 to the first three-way stopcock and the second valve tubing for connecting a first pump to the source of analysis fluid; and a source of analysis fluid. As another example, a user may be provided with a tissue carrier 305, either with or without an already introduced tissue 302 and a first fluid; a first three-way stopcock; a second valve; tubing for connecting the tissue carrier 305 to the first three-way stopcock and the second valve, tubing for connecting a first pump to the source of analysis fluid, and tubing for connecting a second pump to a source of medium fluid; the source of analysis fluid; and the source of medium fluid. As another example, a user may be provided with a tissue carrier 305, either with or without an already introduced tissue 302 and a first fluid; a first three-way stopcock; a second valve; tubing for connecting the tissue carrier 305 to the first three-way stopcock and the second valve, tubing for connecting a first pump to the source of analysis fluid, tubing for connecting a second pump to a source of medium fluid, and tubing for connecting the second valve to an analysis container; the source of analysis fluid; the source of medium fluid; and the analysis container 340. Alternatively, a user could be provided with a tissue carrier 305, either with or without an already introduced tissue and a first fluid; a first three-way stopcock, and a second valve; and any of the other components added to the additionally described groups of components in this paragraph or any other in this disclosure. Additionally, the user may be provided with instructions on how to assemble the components provided. Optionally, the user may be provided with a first pump, a second pump, or both.

In one or more embodiments, the disclosed systems may include a sensor to monitor fluorescence of the analysis fluid. The sensor may detect fluorescence. In one or more embodiments, the sensor may include an excitation light source, a wavelength filter, a detector, or a combination thereof. The sensor may read out a value corresponding to a calculated level of fluorescence. In one or more embodiments, the level of fluorescence may be analyzed to yield an estimated molar concentration of resorufin. In one or more embodiments, the level of fluorescence may be analyzed to yield an estimated cell viability value.

In one or more embodiments, the analysis fluid that may be housed in the source 325 of analysis fluid may be chosen based at least in part on the type of tissue being housed in the tissue carrier 305, the purpose of the tissue, the procedure for which the tissue 302 will be used, and the type of analysis to be done to the tissue, amongst other possible considerations. In one or more embodiments where the tissue 302 being housed in the tissue carrier 305 is a DMEK graft, the analysis fluid may be chosen to analyze the quality of the tissue, the viability of the tissue, other features of the tissue, or any combination thereof.

In one or more embodiments, the analysis fluid may comprise resazurin. As seen in FIG. 1, resazurin has the chemical formula (I) below:

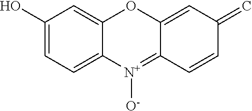 [SMILES: C=c1ccc2c(c1)Oc1cc(O)ccc1[N+]=2[O-]]

Resazurin (7-Hydroxy-3H-phenoxazin-3-one 10-oxide) is a phenoxazine dye that is weakly fluorescent, non-toxic, cell-permeable, and redox-sensitive. Resazurin has a blue to purple color (at pH>6.5) and is used in microbiological, cellular, and enzymatic assays because it may be irreversibly reduced to the pink-colored and highly fluorescent resorufin (7-Hydroxy-3H-phenoxazin-3-one). At circum-neutral pH, resorufin may be detected by visual observation of its pink color or by fluorimetry, with an excitation maximum at 530 nm to 570 nanometers (nm) and an emission maximum at 580 nm to 590 nm.

Resazurin is reduced to resorufin by aerobic respiration of metabolically active cells, and it may be used as an indicator of cell viability. It was first used to quantify bacterial content in milk by Pesch and Simmert in 1929. It may be used to detect the presence of viable cells in mammalian cell cultures.

Resazurin-based assays show excellent correlation to reference viability assays such as formazan-based assays (MTT/XTT) and tritiated thymidine-based techniques. The low toxicity makes it suitable for longer studies, and it has been applied for animal, bacterial, and fungal cells for cell culture assays such as cell counting, cell survival, and cell proliferation.

In one or more embodiments, resazurin may be dissolved in a medium in which the tissue housed in the tissue carrier 305 is stable and maintains its viability. In one or more embodiments where the tissue to be housed in the system 300 is a corneal graft for example, the resazurin may be dissolved in a solution that is capable of preserving the viability of a corneal graft. In general, a solution that is capable of preserving the viability of a corneal graft may include antioxidants and reducing chemicals that do not interfere with any analysis that will be done on the tissue, e.g., the corneal graft. Illustrative types of these solutions may include, for example, Dulbecco's Modified Eagle Medium (DMEM), DMEM/F-12 medium, any endothelial cell medium or balanced salt solution (BSS).

In one or more embodiments, resazurin-based cell viability reagent may be obtained as a commercially available reagent or as a component of a commercially available kit. (e.g., ALAMARBLUE, Trek Diagnostic Systems LLC, Cleveland, OH) and used according to the manufacturer's instructions. For example, ALAMARBLUE cell viability reagent generates a color change and increased fluorescence that may be detected using absorbance (detected at 570 nm and 600 nm) or fluorescence (using an excitation between 530 nm to 560 nm and an emission at 590 nm). To assay for viability, the cell viability reagent can be added to cells in complete media (no wash or cell lysis steps required), incubated for one to four hours, and read using either an absorbance-based or fluorescence-based plate reader. If necessary, longer incubation times may be used for greater sensitivity without compromising cell health.

In one or more embodiments, a solution that includes about 44 μM resazurin, such as a 1× working concentration of an ALAMARBLUE HS Cell Viability Reagent (Trek Diagnostic Systems LLC, Cleveland, OH), 25 milliliters (mL) may be used as the analysis fluid.

The medium fluid may similarly include a solution that is capable of preserving the viability of the tissue being housed in the system. For example, in general, a solution that is capable of preserving the viability of a corneal graft may include antioxidants and reducing chemicals that do not interfere with any analysis that will be done on the tissue, e.g., the corneal graft. Illustrative types of these solutions may include, for example, Dulbecco's Modified Eagle Medium (DMEM), Life4° C. (Numedis, Inc., Isanti MN), or OPTISOL GS (Bausch & Lomb Inc., Bridgewater, NJ). In one or more embodiments, the analysis fluid and the medium fluid may be substantially the same with the exception being that the analysis fluid contains one or more than one chemical entity that allows the analysis to be performed to take place. In one or more embodiments, the analysis fluid and the medium fluid need not be substantially the same and may include two entirely different solutions, the analysis fluid additionally including one or more than one chemical entity that allows the analysis to be performed to take place on the tissue being contained in the tissue carrier.

Methods disclosed herein may include steps of introducing analysis fluid, medium fluid into the tissue carrier. The steps of introducing fluids (e.g., analysis fluid and medium fluid) into the tissue carrier may be accomplished through use of the first and second pumps. In one or more embodiments, the first and the second pumps are the same kind of pumps, and in one or more embodiments, the first and the second pumps are different kinds of pumps. In one or more embodiments, both the first and the second pumps are peristaltic pumps, for example. The flow rate of the liquids into (and out of) the tissue carrier may depend at least in part on the type of tissue being housed in the tissue carrier, the volume of the tissue carrier, the type and construction of the tissue carrier, other parameters relevant to the analysis being undertaken on the tissue, other factors not disclosed herein, or combinations thereof.

In one or more embodiments where the tissue is a corneal graft, such as a DMEK graft, the flow rate of the analysis fluid, the medium fluid, or both into (and out of for example) the tissue carrier may be chosen based on considerations including, for example, the flow rate should not allow the corneal graft to be dislodged and/or allow the graft to exit the tissue carrier, the flow rate chosen should be sufficient to afford necessary influx of the analysis fluid, the medium fluid, or both, into and out of the tissue carrier, or combinations thereof. In one or more embodiments, useful pumps will be those that may produce flow rates of not less than 50 μL/minute, not less than 75 μL/minute, or not less than 100 μL/minute; flow rates of not greater than 750 μL/minute, not greater than 600 μL/minute, or not greater than 500 μL/minute; or any combinations thereof. In one or more embodiments, a flow rate of about 300 μL/minute may be utilized.

Disclosed herein are methods that may include steps of providing a tissue carrier having a tissue and a first fluid contained therein. Introduction of the tissue into the tissue carrier may be undertaken by the same or a different entity than is carrying out the remainder of the method, may be accomplished at the same or a different physical location where the remainder of the method is being carried out, may be accomplished any time before completion of the remaining method steps, or any combination thereof. In one or more embodiments, a tissue carrier may be obtained that has a tissue and a first fluid already previously introduced therein. In one or more embodiments, a tissue carrier and necessary components for forming a disclosed system may be obtained and a user may assemble the components of the disclosed system into its end form (e.g., a tissue carrier in fluid communication with an analysis fluid source and a medium fluid source via a first three-way stopcock, and a second valve). In one or more embodiments, a tissue carrier and necessary components for forming a disclosed system may be obtained as a kit and a user may assemble the components of the disclosed system into its end form (e.g., a tissue carrier in fluid communication with an analysis fluid source and a medium fluid source via a first three-way stopcock, and a second valve)

Disclosed methods may also include a step of introducing a second fluid into the tissue carrier. This step may include actuation of the first three-way stopcock for example. In one or more embodiments, this step could include more specifically introducing an analysis fluid into the tissue carrier. The amount of the second fluid introduced into the tissue carrier may depend on a number of factors, including for example, any of the factors discussed above with respect to the pump rates, the volume of the tissue carrier, etc. In one or more embodiments, where the tissue carrier is a type of modified Jones tube and the tissue is a DMEK graft, a fixed amount of fluid may be introduced, for example not less than 3 milliliters (mL), not less than 5 mL, or not less than 6 mL; not greater than 15 mL, not greater than 12 mL, or not greater than 10 mL; or any combination thereof.

In one or more embodiments, disclosed methods may also include a step of incubating the tissue in the second fluid, e.g., the analysis fluid. The time that the second fluid is incubated in the tissue carrier may depend at least in part on the identity of the second fluid, the chemical entity in the second fluid that is responsible for the analysis of the tissue, the size of the tissue, the volume of the tissue carrier, whether or not the tissue carrier is being subjected to any mechanical manipulation, other factors not disclosed herein, or any combination thereof. In one or more embodiments where the tissue is a DMEK graft, the tissue may be incubated in the second fluid for a time of not less than 20 minutes, not less than 25 minutes, or not less than 30 minutes; or not more than 90 minutes, not more than 75 minutes, or not more than 60 minutes; or any combination thereof. The temperature of incubation may depend on the identity of the second fluid, the chemical entity in the second fluid that is responsible for the analysis of the tissue. In some cases, the graft can be incubated at 4° C., 25° C., 30° C., 35° C., 37° C., or 40° C.

In one or more embodiments, at least a portion of the second fluid may be removed from the tissue carrier. In one or more embodiments, at least a portion (which may be the same portion, the same volume as, or both) of the second fluid removed from the tissue carrier may be collected in the analysis container 340. In one or more embodiments, an amount of the second fluid that is smaller than the amount of the second fluid removed from the tissue carrier may be collected in the analysis container 340 (e.g., a small amount of fluid may be flushed out the second valve and additional connective tubing before a sample is collected for analysis. In one or more embodiments, not less than 0.3 mL of the second fluid may be collected for analysis, not greater than 10 mL of the second fluid may be collected for analysis.

In one or more embodiments, a third fluid may be introduced into the tissue carrier. In one or more embodiments, the third fluid may be the medium fluid from the source of medium fluid, for example.

In one or more embodiments, disclosed methods may also include a step of measuring the presence of resorufin in the collected second fluid. In one or more embodiments, measuring the presence of resorufin in the collected second fluid may be accomplished by measuring the fluorescence or absorbance of the second fluid. In one or more embodiments, a fluorescence intensity of at least a portion of the analysis medium removed from the carrier after a period of incubation may be determined using a fluorescence plate reader (for example, a SYNERGY HTX plate reader, BioTek Instruments, Inc., Winooski, VT) to determine the resorufin produced by live cells within the tissue.

In one or more embodiments, disclosed methods may additionally include a step of correlating the measured fluorescence or absorbance to the number of viable cells in the tissue sample in the tissue carrier.

In some disclosed methods, the tissue carrier may be subjected to mechanical manipulation during, before, after, or some combination thereof any of the additionally performed steps. The mechanical manipulations may serve to aid some of the other noted steps to occur, may serve to hasten some of the additional steps, may serve to aid in the effectiveness of the overall method, may serve to aid in the effectiveness of one or more steps in the overall method, may serve to aid in the reproducibility of the overall method, may serve to aid in the reproducibility of one or more steps in the overall method, or any combination thereof. In some specific embodiments, mechanical manipulations may serve to prevent or minimize the possibility of the tissue being prematurely removed or released from the tissue carrier during one or more of the other steps. In one or more embodiments, mechanical manipulations may serve to ensure complete perfusion of the tissue with analysis fluid or medium fluid, or complete exchange or analysis fluid with medium fluid, or complete exchange of medium fluid with analysis fluid. Different types of mechanical manipulations that may be undertaken may include, but are not limited to, for example, rotation of the tissue carrier, tilting of the axis of the tissue carrier, back and forth movements of the tissue carrier, or any combination thereof.

### EXEMPLARY EMBODIMENTS

Embodiment 1. A composition comprising a corneal tissue carrier,
wherein the corneal tissue carrier comprises a corneal tissue sample and a fluid;
wherein the fluid comprises resazurin.

Embodiment 2. The composition of embodiment 1, wherein the corneal tissue carrier comprises a modified Jones tube. The modified Jones tube may be a Straiko-type modified Jones tube, a LEITR-type modified Jones tube, or another type of modified Jones tube not described herein. The modified Jones tube may be coated with a polymer.

Embodiment 3. The composition of embodiments 1 or 2, wherein the corneal tissue sample comprises a corneal endothelial cell graft

Embodiment 4. The composition of any one of embodiments 1 to 3, wherein the fluid comprises a corneal preservation solution.

Embodiment 5. The composition of any one of embodiments 1 to 4, wherein the concentration of resazurin in the fluid is in a range of 10 μM to 500 μM, 20 μM to 200 μM, or 40 μM to 100 μM.

Embodiment 6. The composition of any one of embodiments 1 to 5, wherein the volume of the fluid is in a range of 50 μL to 800 μL, 100 μL to 600 μL, or 200 μL to 400 μL.

Embodiment 7. A method comprising:
providing a corneal tissue carrier comprising a corneal tissue sample and a first fluid; introducing a second fluid comprising resazurin into the corneal tissue carrier;
incubating the corneal tissue carrier comprising the corneal tissue sample and the second fluid comprising resazurin;
removing the second fluid from the corneal tissue carrier; and collecting at least a portion of the second fluid.

Embodiment 8. The method of embodiment 7, wherein the corneal tissue carrier comprises a modified Jones tube. The modified Jones tube may be a Straiko-type modified Jones tube, a LEITR-type modified Jones tube, or another type of modified Jones tube not described herein. The modified Jones tube may be coated with a polymer.

Embodiment 9. The method of embodiments 7 or 8, wherein the corneal tissue sample comprises a corneal endothelial cell graft.

Embodiment 10. The method of any one of embodiments 7 to 9, wherein the first fluid comprises a corneal preservation solution.

Embodiment 11. The method of any one of embodiments 7 to 10, wherein the second fluid comprises resazurin at a concentration in a range of 10 μM to 500 PM, 20 μM to 200 μM, or 40 μM to 100 μM.

Embodiment 12. The method of any one of embodiments 7 to 11, wherein the second fluid is introduced at a flow rate in a range of 10 μL/minute to 1,000 μL/minute, 50 μL/minute to 800 μL/minute, or 100 μL/minute to 500 μL/minute.

Embodiment 13. The method of any one of embodiments 7 to 12, wherein the volume of second fluid introduced is a fixed volume.

Embodiment 14. The method of any one of embodiments 7 to 13, wherein the second fluid comprises DMEM.

Embodiment 15. The method of any one of embodiments 7 to 14, wherein introducing the second fluid comprises removing the first fluid from the corneal tissue carrier.

Embodiment 16. The method of any one of embodiments 7 to 15, wherein the corneal tissue sample and the second fluid comprising resazurin are incubated for at least 30 minutes to 1 hour.

Embodiment 17. The method of any one of embodiments 7 to 16, wherein removing the second fluid from the corneal tissue carrier comprises introducing a third fluid.

Embodiment 18. The method of embodiment 17, wherein the third fluid comprises a corneal preservation solution.

Embodiment 19. The method of embodiments 17 or 18 wherein the third fluid is introduced at a flow rate in a range of 10 μL/minute to 1,000 μL/minute, 50 μL/minute to 800 μL/minute, or 100 L/minute to 500 μL/minute.

Embodiment 20. The method of any one of embodiments 7 to 19, wherein the method further comprises measuring the presence of resorufin in the second fluid.

Embodiment 21. The method of embodiment 20, wherein the method further comprises correlating the presence of resorufin to the number of viable cells in the corneal tissue sample.

Embodiment 22. The method of any one of embodiments 7 to 21, wherein the method further comprises measuring the fluorescence or absorbance of the second fluid.

Embodiment 23. The method of embodiment 22, wherein the method further comprises correlating the fluorescence or absorbance to the number of viable cells in the corneal tissue sample.

Embodiment 24. The method of any one of embodiments 7 to 23, wherein removing the second fluid from the corneal tissue carrier comprises manipulating the corneal tissue carrier.

Embodiment 25. A system, comprising:
a corneal tissue carrier comprising an inlet, an outlet, and a body portion tapered from the inlet toward the outlet;
   an input valve coupled to the corneal tissue carrier inlet and configured to control input of a first fluid and input of a second fluid to the corneal tissue carrier, wherein one of the first and second fluids comprises resazurin;
   an output valve coupled to the corneal tissue carrier outlet and configured to control output of fluid from the corneal tissue carrier; and
   a manipulation system coupled to the corneal tissue carrier and configured to control movement of the corneal tissue carrier.

Embodiment 26. The system of embodiment 25, wherein the corneal tissue carrier comprises a modified Jones tube.

Embodiment 27. The system of embodiments 25 or 26, wherein the corneal tissue carrier stores a corneal endothelial cell graft.

Embodiment 28. The system of any one of embodiments 25 to 27, wherein the input valve comprises a first input coupled to a first fluid reservoir and a second input coupled to a second fluid reservoir and the input valve is configured to input one fluid to the corneal tissue carrier at a time.

Embodiment 29. The system of embodiment 28, wherein the first input is coupled to a pump.

Embodiment 30. The system of embodiments 28 or 29, wherein the second input is coupled to a pump.

Embodiment 31. The system of any one of embodiments 25 to 30, wherein the output valve is coupled to a receptacle configured to receive output fluid comprising resazurin.

Embodiment 32. The system of any one of embodiments 25 to 31, further comprising a sensor system configured to monitor fluorescence of the fluid comprising resazurin in the corneal tissue carrier.

Embodiment 33. The system of any one of embodiments 25 to 32, where in the manipulation system is configured to minimize premature release of the tissue from the tissue carrier.

Embodiment 34. A corneal tissue carrier, comprising:
an inlet configured to receive a first fluid and a second fluid comprising resazurin; an outlet; and
   a body portion tapered from the inlet toward the outlet, wherein the tapering directs fluid flow from the inlet to the outlet in response to manipulation of the body portion.

Embodiment 35. The system of embodiment 34 wherein the corneal tissue carrier comprises a modified Jones tube. The modified Jones tube may be a Straiko-type modified Jones tube, a LEITR-type modified Jones tube, or another type of modified Jones tube not described herein. The modified Jones tube may be coated with a polymer.

Embodiment 36. The system of embodiments 34 or 35, wherein the corneal tissue carrier stores a corneal endothelial cell graft.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to”, “at most”, “not more than”, or “not greater than” a number (for example, up to 50) includes the number (for example, 50). Herein, “at least”, or “not less than” a number (for example, at least 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

Any reference to standard methods (e.g., slit lamp microscopy, specular microscopy, Gabor-Domain OCT) refer to the most recent available version of the method at the time of filing of this disclosure unless otherwise indicated.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### EXAMPLES

The present invention is illustrated by the following examples. It is to be understood that the particular examples, materials, amounts, and procedures are to be interpreted broadly in accordance with the scope and spirit of the disclosure as set forth herein.

All reagents, starting materials, and solvents used in the following examples were purchased from commercial suppliers (such as Sigma Aldrich, St. Louis, MO) and were used without further purification unless otherwise indicated.

### Example 1—Using a Disclosed System to Evaluate the Endothelial Cell Loss (ECL) of a Descemet's Membrane Endothelial Keratoplasty (DMEK) Graft

A DMEK graft was prepared as follows. In order to reduce sample variation, endothelial cells were first harvested from human donor corneas and the cells were seeded at 2500-3000 cells per mm² onto a denuded endothelium (with cells removed) of mate cornea pairs. After 24 hours, the endothelial cells were attached to the endothelium completely and uniformly. The endothelium with the seeded cells was subjected to a DMEK peeling procedure to generate DMEK grafts. The grafts were then loaded into two different LEITR glass tubes. Tube A was a LEITR modified Jones tube and Tube B was a LEITR modified Jones tube coated with 20K PEG). Each tube was then perfused with ALAMARBLUE (Trek Diagnostic Systems LLC, Cleveland, OH) and the grafts were then incubated at 37° C. for one hour. After the one-hour incubation, the glass tubes with grafts were perfused and 2 mL solution was collected. These are the cell viability “Before ejection” samples.

The DMEK grafts were then ejected from the glass tubes into a six-well tissue culture plate and incubated in 2 mL ALAMARBLUE solution (Trek Diagnostic Systems LLC, Cleveland, OH) at 37° C. for another hour. After the second one-hour incubation, the solutions were collected (cell viability “After ejection”) and measured for their fluorescence intensity.

The ECL caused by graft ejection from the glass tubes were determined according to the reduction of cell viability (After rejection divided by Before ejection).

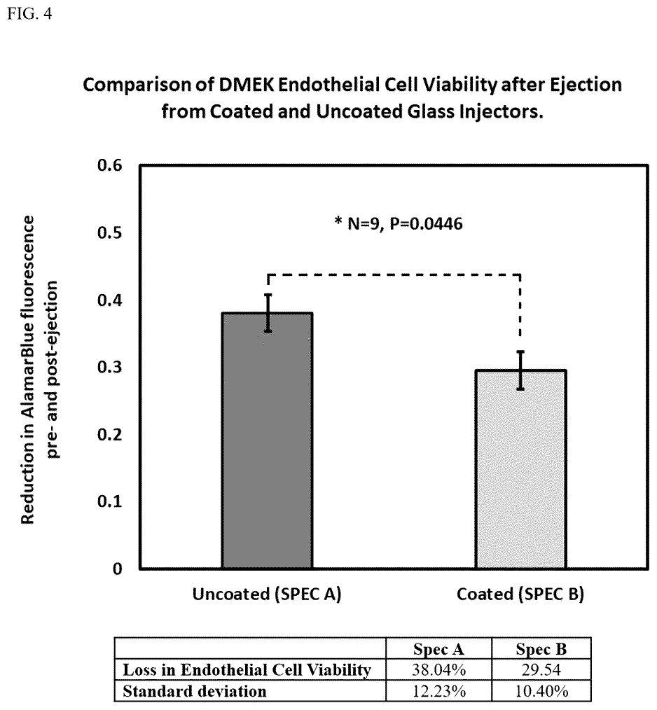

As shown in FIG. 4, graft ejection caused 38% ECL for SPEC A and 29% ECL for SPEC B. These numbers are higher than previously reported graft ejection ECL rates (25%) measured using Calcein-AM to stain dead cells (Tran et al., Cornea. 37(8):1075-1080, 2018). These results indicate that ejection-related cell damage/death is underestimated using Calcein-AM staining. Therefore, better cell damage/death evaluation methods such as the systems or methods disclosed herein are desirable. Disclosed systems may also be used for product development and optimization of newly modified tissue carriers.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1-6. (canceled)

7. A method comprising:
providing a corneal tissue carrier comprising:
a corneal tissue sample;
a first fluid solution;
introducing a second fluid solution comprising resazurin into the corneal tissue carrier;
incubating the corneal tissue carrier comprising the corneal tissue sample and the second fluid solution;
removing the second fluid solution from the corneal tissue carrier; and
collecting at least a portion of the second fluid solution.

8. The method of claim 7, wherein the corneal tissue carrier comprises a modified Jones tube.

9. The method of claim 7, wherein the corneal tissue sample comprises a corneal endothelial cell graft.

10. The method of claim 7, wherein the first fluid solution comprises a corneal preservation solution.

11. The method of claim 7, wherein the second fluid solution comprises resazurin at a concentration in a range of 40 μM to 100 μM.

12. The method of claim 7, wherein a volume of the second fluid solution introduced is a fixed volume.

13. The method of claim 7, wherein removing the second fluid solution from the corneal tissue carrier comprises introducing a third fluid solution, wherein the third fluid solution comprises a corneal preservation solution.

14. The method of claim 7, wherein the method further comprises:
measuring the concentration of resorufin in the second fluid solution; and
correlating the concentration of resorufin to a number of viable cells in the corneal tissue sample.

15. A system comprising:
a corneal tissue carrier comprising an inlet, an outlet, and a body portion tapered from the inlet toward the outlet;
an input valve coupled to the corneal tissue carrier inlet and configured to control input of a first fluid solution and input of a second fluid solution to the corneal tissue carrier, wherein the first fluid solution comprises resazurin;
an output valve coupled to the corneal tissue carrier outlet and configured to control output of fluid solution from the corneal tissue carrier; and
a manipulation system coupled to the corneal tissue carrier and configured to control movement of the corneal tissue carrier.

16. The system of claim 15, wherein the corneal tissue carrier comprises a modified Jones tube.

17. The system of claim 15, wherein the corneal tissue carrier stores a corneal endothelial cell graft.

18. The system of claim 15, wherein the input valve comprises a first input coupled to a first fluid reservoir and a second input coupled to a second fluid reservoir and the input valve is configured to input one fluid solution to the corneal tissue carrier at a time.

19. The system of claim 18, wherein the first input is coupled to a first pump, or the second input is coupled to a second pump, or both.

20. The system of claim 15, further comprising a sensor system configured to monitor fluorescence of resorufin in the fluid solution in the corneal tissue carrier.

21. The system of claim 15, wherein the second fluid solution comprises a corneal preservation fluid.
